(19) United States
(12) Patent Application Publication
(10) Pub. No.: US 2005/0222452 A1
(43) Pub. Date: Oct. 6, 2005

(54) Process for preparing dioxy-functionalized propane compounds

(75) Inventors: Raghunath Vitthal Chaudhari, Pune (IN); Yogesh Laxman Borole, Pune (IN); Shubhangi Bhalchandra Umbarkar, Pune (IN)

Correspondence Address: William R. Evans;Ladas & Parry, 26 West 61 Street, New York, NY, 10023, US

(73) Assignee: Council of Scientific and Industrial Research

(21) Appl. No.: 10/814,859

(22) Filed: Mar. 31, 2004

(51) Int. Cl. C07C 69/76; C07C 29/14

(52) U.S. Cl. 560/232; 568/858

(57) ABSTRACT

The present invention relates to a process for preparing, dioxy functionalised propane compounds using cobalt and/or rhodium catalysts. More particularly, the present invention relates to a process for the preparation of the dioxy functionalised propane compounds of the formula X—CH₂—CH(Y)—CH₂-Z, wherein X=R—C(═O)—O— or H or —OH, Y and Z=—H or —OH or —C(═O)H, and wherein at a time, only one of X, Y and Z is —H, in high yields from vinyl carboxylates of formula R—C(═O)—O—CH═CH₂ wherein, R=substituted or unsubstituted alkyl or aryl group.

### FIELD OF INVENTION

The present invention relates to a process for preparing dioxy functionalised propane compounds using cobalt and/or rhodium catalysts. More particularly, the present invention relates to a process for the preparation of the dioxy funtionalised propane compounds of the formula X—CH₂—CH(Y)—CH₂-Z, wherein X=R—C(═O)—O— or H or —OH, Y and Z =—H or —OH or —C(═O)K and wherein at a time, only one of X, Y and Z is —H, in high yields from vinyl carboxylates of formula R—C(═O)—O—CH═CH₂ wherein, R═substituted or unsubstituted alkyl or aryl group.

### BACKGROUND OF THE INVENTION

Dioxy-functionalized compounds such as propanediols are useful products having a variety of applications. 1,2-propanediol (1,2-PDO) is used as a coolant and antifreeze agent, and also to prepare polypropylene glycol. 1,3-propanediol (1,3-PDO) is a useful intermediate in production of polyesters for fibers and films as well as starting material for synthesis of cyclic compounds. 1,3-PDO is also a useful intermediate in the production of polyurethanes.

Several methods have already been suggested for the preparation of 1,2-PDO as well as 1,3-PDO. 1,2PDO is obtained for example by hydrolysis of propylene oxide. 1,3-PDO is obtained either by acrolein hydration route or by ethylene oxide hydroformylation route or by biotransformation of a carbohydrate source. In U.S. Pat. No. 2,434,110 acrolein is hydrated in the presence of an acidic catalyst to form 3-hydroxypropanal. The reaction takes place preferably at an elevated temperature using a 5 to 30% by weight solution of acrolein in water and an acid such as sulphuric acid phosphoric acid or acidic salts of these acids, acidic resins as the catalyst. The reaction mixture obtained during the hydration is hydrogenated, preferably after removal of non-reacted acrolein, in the presence of customary hydrogenation catalysts. DE-A3926136, DE-A 4038192, U.S. Pat. No. 2,638,479, U.S. Pat. No. 3,536,763, U.S. Pat. No. 5,015,789, U.S. Pat. No. 5,093,537, U.S. Pat. No. 5,171,898, U.S. Pat. No. 5,276,201, and U.S. Pat. No. 5,364,987 also describe processes for production of 1,3-propanediol from acrolein. The drawbacks of the above referred documents are use of acrolein; which is corrosive and highly toxic material, relatively low reaction rates, lesser product yields and unwanted side reactions at higher acrolein conversion. Therefore, this approach has not particularly been attractive for making 1,3-propanediol in large quantities.

The preparation of 1,3-PDO by hydroformylation of epoxides utilizing cobalt carbonyl or rhodium carbonyl complexes as catalysts is disclosed in many documents. References is made to U.S. Pat. Nos. 3,463,819, 5,304,691, 5,344,993, 5,304,686, 5,545,766, 5,723,389, 5,981,808, 6,323,374B1 and 5,463,145, describe cobalt catalyzed hydroformylation of ethylene oxide. U.S. Pat. No. 4,873,378, EP 0257967, U.S. Pat. Nos. 5,210,318, 4,935,554, 5,030,766 and 5,053,562 describes processes for 1,3-PDO by ethylene oxide hydroformylation with rhodium based catalyst systems, wherein 3-hydroxypropanal, obtained by ethylene oxide hydroformylation, is hydrogenated to 1,3-PDO. Drawbacks of above processes are the use of ethylene oxide which is highly flammable and toxic starting material, induction period in rhodium catalyzed hydroformylation, very high catalyst to substrate ratio in cobalt catalyzed hydroformylation and side reactions such as ethylene oxide isomerisation to acetaldehyde.

Reference is made to the U.S. Pat. Nos. 5,599,689, 5,686,286 and 6,025,184 wherein a process for the biotransformation of a carbohydrate carbon source to 1,3-PDO using mixed yeast and bacterial cultures is described. The drawbacks of these processes are low conversion and selectivity, product recovery problems etc.

U.S. Pat. Nos. 5,364,984, 5,945,510, 5,786,524, and 6,342,464 teach preparation of 1,3-propanediol by hydrogenation of 3-hydroxypropanal obtained either by acrolein hydration or by ethylene oxide hydroformylation. Mainly Ni or Ru catalysts are used in mostly trickle bed reactors. The drawback associated with this type of 13hydrogenation is poisoning of the catalyst by impurities in the 3-hydroxypropanal feed and the impurities generated during the reaction.

Only a few processes have become known whereby propanediols are produced simultaneously from one raw material. References is made to U.S. Pat. No. 4,642,394, wherein both 1,2- and 1,3-propanediol can be produced by reaction of glycerol with carbon monoxide and hydrogen in an organic solvent in the presence of a homogeneous catalyst system consisting of, for example, tungstic acid and a rhodium compound. The drawback of this process is low yields of 1,2-PDO and 1,3-PDO, which in each case scarcely exceeds 20%; moreover, glycerol must be reacted in solution in an amine or amide so that aqueous glycerol solution cannot be used.

Reference is made to U.S. Pat. No. 4,642,394 which describes a process for the simultaneous production of 1,2 and 1,3 propanediol from glycerol, involving first dehydration of glycerol to prepare acrolein and hydroxyacetone, hydration of acrolein to 3-hydroxypropanal and hydrogenation of both hydroxyacetone and 3-hydroxypropanal to 1,2-PDO and 1,3-PDO respectively. This process carries disadvantages of acrolein hydration route. Also the temperature required (˜300° C.) for glycerol dehydration is very high and overall selectivity to propanediols is less (70%).

The first reference for vinyl acetate monomer (VAM) hydroformylation dates back to 1949, wherein Adkins et al (J. Amer. Chem. Soc., 71, 3051 (1949)) (incorporated herein as a reference) have carried out hydroformylation of VAM with cobalt carbonyl as a catalyst at 125° C. and under 4600 psi pressure of 1:1 mixture of carbon monoxide and hydrogen. They obtained ˜35% selectivity for 3-acetoxy propane and 65% selectivity for 2-acetoxy propanal based on total acetoxy propanals obtained. The pressures used by Adkins et al are very high and conversion is only ˜70%. There are few reports on rhodium-catalyzed hydroformylation of vinyl acetate. References is made to DE 1225627, U.S. Pat. No. 4,072,709, Tetrahedron: Asymmetry vol. 3 No. 5, pp. 583, 1992 etc., wherein rhodium catalyzed hydroformylation of vinyl acetate invariably gives >90% selectivity for 2-acetoxypropanal. Reference is made to U.S. Pat. No. 4,072,709 wherein VAM hydroformylation route for lactic acid production is described whereby VAM is first hydroformylated to 2-acetoxypropanal, which on oxidation and hydrolysis gives lactic acid. In ‘The asymmetric hydroformylation of vinyl acetate’, Bulletin of the Chemical Society of Japan, Vol. 52 (9), 2735-2736, (1979) (incorporated herein as a reference), Watanabe discloses VAM hydroformylation with rhodium catalyst and optically active ligand to produce optically active 2-acetoxypropanal. In the same document, Watanabe also describes hydrogenation and hydrolysis of 2-acetoxypropanal with lithium aluminum hydride and 1 M HCI to produce optically active 1,2-propanediol. However, Watanabe could get only 2-acetoxypropanal with rhodium catalyst and the hydrogenation and hydrolysis is carried out with hazardous reagents. To the best of our knowledge there is no report on simultaneous production of both 1,2- and 1,3-PDO through VAM hydroformylation and catalytic hydrogenation and hydrolysis.

### OBJECT OF THE INVENTION

The main object of the present invention is to provide a process for dioxy functionalized propane compounds using cheap, readily available, and non-corrosive reactants such as vinyl carboxylates and using recyclable catalysts for hydroformylation, hydrogenation, and hydrolysis and which obviates the drawbacks detailed above.

Another object is to provide a process for propanediols production which obtains sufficient yields of useful intermediates such as 2- and 3-acetoxypropanals, 2- and 3-hydroxypropanals, 2- and 3-acetoxypropanols.

It is another object of the invention to provide a process for the preparation of propanediols which utilizes efficient conversion and separation procedures so as to be technologically and economically practical in terms of cost and efficiency factors with respect to raw materials and conversion procedures.

Another object of the present invention is to provide a process for propanediols production, with simple and inexpensive catalyst-product separation procedures.

### SUMMARY OF THE INVENTION

Accordingly the present invention provides a process for preparing dioxy-functionalized propane compounds of formula X—CH₂—CH(Y)—CH₂-Z wherein X =R—C(═O)—O— or H or —OH, Y and Z =—H or —OH or —C(═O)H, and wherein at a time only one of X Y and Z is —H, which comprises:
a. contacting a vinyl carboxylate with carbon monoxide and hydrogen in a solvent in the presence of transition metal catalyst, to obtain an intermediate product mixture comprising of 3-carboxypropanal and 2-carboxypropanal;
b. adding water to the intermediate product mixture of 3-carboxypropanal and 2-carboxypropanal to extract the carboxypropanal; into water to obtain an aqueous phase comprising of carboxypropanols and an organic phase comprising of transition metal catalyst and separating the aqueous phase from the organic phase,
c. contacting the aqueous phase comprising of carboxypropanols with hydrogen in presence of a heterogeneous hydrogenation catalyst to obtain a hydrogenation product mixture comprising of 3-carboxypropanol and 2-carboxypropanol in aqueous phase and separating the hydrogenation catalyst from the aqueous phase;
d. contacting the aqueous phase obtained in (c) comprising of carboxypropanols with a hydrolysis catalyst at a temperature within the range of 30° C. to 120° C. at least during a portion of a hydrolysis step to provide a product mixture comprising of 1, 3- and 1, 2-propanediol and a corresponding carboxylic acid;
e. separating and recovering carboxylic acid, 1, 3- and 1, 2-propanediols from the aqueous mixture by distillative separation.

In one embodiment of the invention, the organic phase obtained in step (b) is recycled to step (a).

In another embodiment of the invention, steel (a) above is carried Out in the presence of a promoter or a ligand and at a temperature in the range of 50° C. to 140° C. and a pressure in the range of 500 psi to 5000 psi.

In another embodiment of the invention, step (b) is carried out at a temperature of not more than 100° C.

In another embodiment of the invention, the aqueous phase of carboxypropanals is separated from the organic phase comprising transition metal by phase separation.

In another embodiment of the invention, step (c) is carried out at a minimum temperature of 40° C. and at least for a period of 30 min, while optionally increasing the reaction temperature to 80° C. and hydrogen pressure of at least 200 psi.

In another embodiment of the invention, the aqueous phase comprising carboxypropanols in step (c) is separated from the hydrogenation catalyst by filtration, decantation or centrifugation.

In another embodiment of the invention, the transition metal catalyst used in step (a) comprises a Group VIII transition metal catalyst selected from the group consisting of rhodium, ruthenium, iridium, cobalt, palladium and nickel.

In another embodiment of the invention, the transition metal catalyst is selected from the group consisting of cobalt carbonyl rhodium carbonyl and any mile thereof

In another embodiment of the invention, the transition metal catalyst is modified by a ligand comprising an organic compound of a Group V element selected from the group consisting of triaryl- phosphines, trialkyl-phosphines, arsines and amines.

In yet another embodiment of the invention, the catalyst of step (a) is used in the form of metal, supported metal, hydroxide, oxide, carbonate, sulfate, acetylacetonate, salt of a carboxylic acid, and an aqueous salt solution.

In yet another embodiment of the invention, the catalyst of step (a) is used directly in the form of cobalt carbonyl or rhodium carbonyl selected from the group consist of dicobalt octacarbonyl, cobalt hydridocarbonyl, tetrarhodium dodecacarbonyl, and dicarbonylacetylacetatonatorhodium.

In another embodiment of the invention, the catalyst of step (a) is used in the form of an organometallic complex of the transition metals and ligands selected from the group consisting of HRh(CO)(PPh₃)₃ and HCo(CO)₃(PBu₃).

In yet another embodiment of the invention, the catalyst of step (a) in carbonyl form is formed in situ through the reaction of the metal with H2 and CO, and at a temperature of at least 50° C. and a carbon monoxide partial pressure of at least about 100 psi.

In yet another embodiment of the invention, the temperature for in situ catalyst formation is in the range of 120° C. to 200° C., and the CO pressures is at least about 500 psi.

In yet another embodiment of the invention, high surface area activated carbon or zeolite, such as those containing or supporting platinum or palladium metal are added to the reactor to form cobalt carbonyl formation from noncarbonyl precursors.

In another embodiment of the invention, the amount of transition metal present in the reaction mixture is in the range of 0.01 to 1 wt %, preferably 0.05% to 0.3 wt %, based on the weight of the reaction mixture.

In another embodiment of the invention, the cobalt catalyst is present in the range of 0.005 to 0.5 wt %, preferably 0.01 to 0.1 wt % based on the weight of the reaction mixture.

In another embodiment of the invention the hydroformylation catalyst in step (a) comprises a cobalt complex selected from the group consisting of cobalt acetate, cobalt hydroxide, cobalt oxide, cobalt carbonate, cobalt sulfate, cobalt acetylacetonate, cobalt carboxylate, dicobalt octacarbonyl cobalt hydridocarbonyl and HCo(CO)₃(PBu₃).

In another embodiment of the invention the hydroformylation catalyst in step (a) comprises a rhodium complex selected from the group consisting of rhodium acetate, rhodium hydroxide, rhodium oxide, rhodium carbonate, rhodium sulfate, rhodium acetylacetonate, rhodium carboxylate, tetrarhodium dodecacarbonyl, dicarbonylacetylacetatonatorhodium and an organometallic complexes of the transition metals and a ligand comprising HRh(CO)(PPh₃)₃.

In another embodiment of the invention, the cobalt carbonyl catalyst used in step (a) includes a lipophillic amine promoter in an amount effective to promote the hydroformylation reaction to acetoxypropanals, generally an amount within the range of 0.01 to 9 moles, preferably within the range of 0.6 to 3 moles based on cobalt.

In another embodiment of the invention, the lipophillic amine is of the formula: 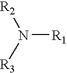
wherein each of R₁, R₂ and R₃ are independently selected from hydrogen and/or unsubstituted and non-interfering substituted C₁-C₂₅ hydrocarbons.

In another embodiment of the invention, the amine is selected from the group consisting of dimethyl amine, diethyl amine, n-butyl amine, di-n-butyl amine, sec-butyl amine, di-sec-butyl amine, n-hexyl amine, di-n-hexyl amine, octyl amine, di(2-ethylhexyl) amine, dodecyl amine, steryl amine, allyl amine, diallyl amine, crotyl amine, dicrotyl amine, cyclopentyl amine, dicyclopentyl amine, dicyclohexyl amine, benzyl amine, dibenzyl amine, phenyl ethyl amine, diphenyl amine, cinnamyl amine, dicinnamyl amine, aniline, o-, m- and p- toludines, 1,2,3-xylidine, 1,2,4- xylidines, 1,3,5-xylidines, 1,3,4-xylidine, mesidine, pacudocumidine, monoethyl aniline, benzyl aniline, pyrrole, guanidine, triethyl amine, trimethyl amine, tributyl amine, trihexyl amine, triphenyl amine and diphenyl methyl amine.

In another embodiment of the invention, two of R₁, R₂, and R₃ form a ring structure such as Pyridine and substituted pyridines of the formula 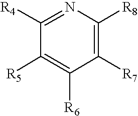
wherein each of R₄, R₅, R₆ and R₇ is independently selected from hydrogen and C₁-C₂₅ hydrocarbons.

In another embodiment of the invention, the promoter is preferably a non-chelating amine of a conjugate acid (pKa about 5-11) selected from the group consisting of tertiary amines including dimethyldodecylamine, pyridine, 4-(1-butylpentyl)pyridine, quinoline, isoquinolein, lipdine and quinaldine, preferably pyridine.

In yet another embodiment of the invention, the vinyl carboxylate is of the formula R—C(═O)—O—CH═CH₂ where R is an organic group selected from the group consisting of an alkyl group, aryl group, an organic hydrocarbyl radical comprising an alkyl group with 1 to 6 carbon atoms, phenyl and tolyl.

In another embodiment of the invention, the vinyl carboxylate is a residue of an organic acid selected from the group consisting of formic acid, acetic acid, propionic acid and benzoic acid.

In yet another embodiment of the invention vinyl carboxylate is vinyl acetate.

In another embodiment of the invention, the vinyl carboxylate is present at the start of the hydroformylation step in a concentration in the range of 0.01% to 95%, preferably 0.5% to 75%, by weight based on the total weight of reactants, catalyst and liquid medium present.

In yet another embodiment of the present invention the carboxylic acid, corresponding to the vinyl carboxylate used, recovered in step (e) is recycled to produce the vinyl carboxylate starting material, In another embodiment of the invention, step (a) is carried out with a cobalt/rhodium catalyst and a ligand comprising a nitrogen containing ligand selected from the group consisting of triphenyl amine, triethyl amine, tricyclohexyl amine, pyridine and substituted pyridine, preferably, 4-methyl pyridine.

In yet another embodiment of the invention the ligand used with the cobalt/rhodium catalyst in step (a) comprises a phosphorous, arsine or stilbine containing ligand selected from the group consisting of triphenyl phosphine, tributyl phosphine, triphenyl arsine and tryphenyl stilbine.

In another embodiment of the invention the ligand used with the cobalt/rhodium catalyst in step (a) comprises a polydentate ligand selected from the group consisting of diphenyl phosphinoethane, diphenyl phosphinopropane, diphenyl phosphinobutane, diphenyl phosphinopentane, bipyridine and terpyridine.

In another embodiment of the invention, the cobalt/rhodium catalyst used in step (a) is used along with a promoter comprising a quaternary ammonium salt selected from the group consisting of tetra alkyl ammonium halide and tetra alkyl ammonium hydroxide.

In another embodiment of the invention, the promoter used in step (a) along with cobalt/rhodium catalyst comprises a quaternary phosphonium salt selected from the group consisting of tetra alkyl phosphonium halide and tetra alkyl phosphonium hydroxide.

In another embodiment of the invention, the ligand used in step (a) along with cobalt/rhodium catalyst comprises an optically active ligand in order to obtain optically active -chiral 2-acetoxy propanal.

In one embodiment of the invention, the solvent used in step (a) comprises an aliphatic hydrocarbon selected from the group consisting of hexane, cyclohexane and decane.

In another embodiment of the invention, the solvent of step (a) comprises an aromatic hydrocarbon selected from the group consisting of benzene, toluene and xylene, preferably toluene.

In yet another embodiment of the invention, the solvent used in step (a) is selected from the group consisting of high molecular weight ethers, polyethers, especially glycol polyethers, and cyclic ethers, such as diethyl ether, methyl-t-butyl ether, ethyl-t-butyl ether, preferably, methyl t-butyl ether.

In yet another embodiment of the invention, the solvent used in step (a) is selected from the group consisting of amides and sulpholanes.

In yet another embodiment of the invention, the solvent used in step (a) comprises a chlorinated solvent selected from the group consisting of dichloromethane, dichloroethane, chlorobenzene and dichlorobenzene.

In another embodiment of the invention, the solvent used in step (a) is selected from the group consisting of an alcohol, a ketone, an ester and mixtures thereof

In another embodiment of the invention, the solvent used in step (a) comprises a solvent blend selected from the group consisting of tetrahydrofuran/toluene, tetrahydrofuran/heptane, and t-butylalcohol/hexane.

In another embodiment of the invention the solvent used in step (a) is a mixture of a non-miscible hydrocarbon solvent comprising toluene and an aqueous solvent compriaing water, enabling the separation of products obtained in aqueous phase during the course of the reaction.

In one embodiment of the invention, the hydroformylation reaction in step (a) is carried out at a temperature of about 140° C., preferably 90° C. to 130° C., most preferably 100° C. to 120° C., and at a pressure within the range of about 500 to about 5000 psi preferably about 1000 to about 3500 psi

In another embodiment of the invention, step (a) is carried out at temperature in the range of 90 to 120° C.

In another embodiment of the invention, the hydrogen and carbon monoxide are introduced in the reaction in a molar ratio in the range of 1:2 to 8:1, preferably 1.5:1 to 5:1.

In another embodiment of the invention CO and H₂ are used in step (a) in a ratio in the range of 4:1 to 1:4.

In another embodiment of the invention, the rhodium catalyzed hydroformylation of vinyl carboxylate includes a phosphorous, nitrogen, arsine or stilbine containing ligand such as monodentate ligands such as triphenyl phosphine, tributyl phosphine, triphenyl amine, triphenyl arsine, triethyl amine, tricyclohexyl amine, pyridine, substituted pyridines such as 4-methyl pyridine, etc or polydented ligands such as diphenyl phosphinoethane, diphenyl phosphinopropane, diphenyl phosphinobutane, diphenyl phosphinopentane, bipyridine, terpyridine.

In another embodiment of the invention, the reaction is carried out in the presence of an optically active ligand in order to obtain optically active 2-acetoxypropanal.

In a further embodiment of the invention, the ratio of the ligand is at least one equivalent of ligand per mole of metal in the catalyst.

In another embodiment of the invention, water is added in step (b) as to provide a water hydroformylated mixture ratio in the range of 1:1 to 1:20, preferably 1:5 to 1:15.

In another embodiment of the invention, the water extraction is preferably carried out at a temperature in the range of 25° C. to 55° C. and under 50 to 300 psi carbon monoxide.

In another embodiment of the invention, hydroformylation is carried out using reverse biphasic system in continuous mode, wherein water is added as a co-solvent during the hydroformylation in step (a) to create a separate phase thereby enabling extraction during the hydroformylation step.

In another embodiment of the invention the aqueous extract containing acetoxypropanals obtained in step (c) is passed through a bed of purifying agents.

In another embodiment of the invention the purifying agent is purifying carbon.

In another embodiment of the invention, hydrogenation of the acetoxypropanals in step (c) to acetoxypropanols is carried out in aqueous solution at an elevated temperature of at least about 40° C., generally in the range of 50° C. to 175° C. and under a hydrogen pressure of at least about 100 psi generally in the range of 200 to 2000 psi.

In yet another embodiment of the invention, hydrogenation is carried out in the presence of a hydrogenation catalyst such as any of those based upon group VIII metals including Ni, Co, Ru, Pt and Pd as well as Cu, Zn, Cr, Au catalysts including bulk, supported and fixed bed forms.

In another embodiment of the invention the hydrogenation catalyst of step (c) is a supported catalyst selected from the group consisting of ruthenium, nickel, cobalt, platinum and palladium catalyst,

In another embodiment of the invention the temperature of the hydrogenation reaction of step (c) is in the range of 40° C. to 90° C.

In another embodiment of the invention the hydrogenation catalyst separated in step (c) is recycled.

In another embodiment of the invention the hydrogenation reaction of step (c) is carried out in a continuous manner.

In another embodiment of the invention the hydrogenation catalyst in step (c) is Ru/Al₂O₃.

In another embodiment of the invention the hydrogenation catalyst in step (c) is Raney Ni.

In another embodiment of the invention the hydrogenation is carried out in the same solvent of hydroformylation, preferably after removal of the hydroformylation catalyst by complexation or using activated charcoal/activated silica.

In another embodiment of the invention the hydrogen pressure of step (c) is in the range of 200 psig to 800 psig.

In another embodiment of the invention, the hydrolysis of step (d) is carried out in the presence of an acid or base catalysts for ester hydrolysis, such as mineral acids, sulphonic acids, ion exchange resins, solid acids such as ZSM-5 etc., heteropolyacids, carboxylic acids, sodium hydroxide, potassium hydroxide.

In another embodiment of the invention, the temperature of the hydrolysis reaction for an ion exchange resin catalyzed hydrolysis reaction is in the range of from room temperature to 120° C., preferably from 50° C. to 80° C.

In another embodiment of the invention, the ion exchange resin used is Amberlite IR-120 cation exchange resin for the hydrolysis of a mixture of 2- and 3-acetoxypropanols to 1,2- and 1,3-propanediols.

In another embodiment of the invention, hydrolysis in step (d) is carried out with 10% HCI

In another embodiment of the invention the hydrolysis step (d) is carried out in a continuous manner.

In another embodiment of the invention the hydrolysis catalyst from step (d) is activated and recycled.

In another embodiment of the invention the carboxylic acid, 1,3-propanediol, and 1,2-propanediol formed are separated by fractional distillation in step (e).

In another embodiment of the invention the hydrolysis in step (d) is carried out before hydrogenation of step (c) in order to obtain 2- and 3- hydroxy propanal.

### DETAILED DESCRIPTION OF THE INVENTION

Hydroformylation processes are well known in the art and have been directed to the production of reaction mixtures comprising substantial amounts of aldehydes and alcohols by the reaction of olefins with carbon monoxide and hydrogen at elevated temperatures and pressures in the presence of certain catalysts. The prior art teaches the use of dicobalt octacarbonyl or its various modified forms as well as carbonyls of other Group VIII metals such as rhodium, ruthenium and iridium which may also be modified by ligands comprised of organic compounds of Group V elements such as triaryl- and trialkyl-phosphines, arsines, amines and the like. Some disadvantages such as less regioselectivity for either normal or branched aldehyde production, expensive catalyst-product separation and the like, are present in the hydroformylation processes. Because of the less regioselectivity, undesired aldehyde along with the desired one is also produced, which in turn affects the overall process economics because of the low yields and costly purification procedures. Whereas, the hydroformylation process in accordance with procedures described herein overcomes the above said disadvantages in an effective way.

It has now been found that 1,3-propanediol can efficiently be produced in accord with the following:

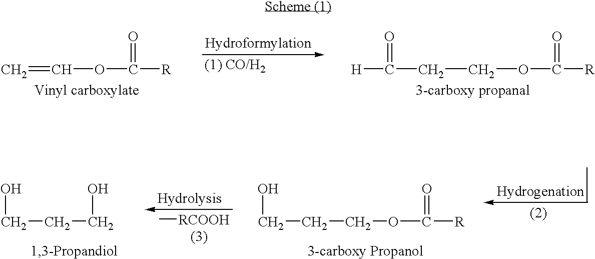

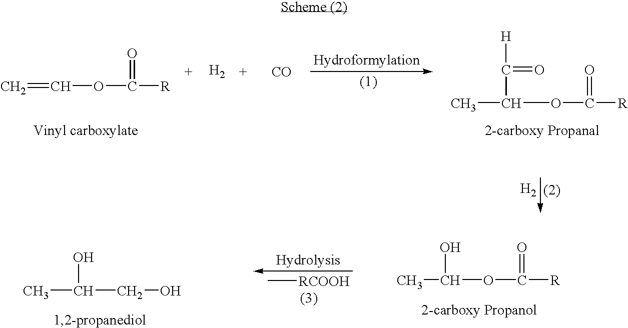

The order of the hydrogenation and hydrolysis can be reversed or both these reactions can be carried out simultaneously.

In the illustrated reactions, the products of VAM (where R is an acetate group) hydroformylation are 3-acetoxypropanal and 2-acetoxypropanal, and in accordance with the present invention it has been found possible to obtain these products in high yields by the use of suitable hydroformylation catalysts. In this type of procedure it is also possible to produce ethyl acetate by VAM hydrogenation or ethylene and acetic acid by VAM hydrolysis, but conditions have been found which give high yields of the acetoxypropanals.

The carboxylate part of the vinyl carboxylate gets converted into carboxylic acid during the process of producing propanediols. As used herein, the terms propanediols, acetoxypropanals, acetoxypropanols, hydroxypropanals refer not only to the monomeric forms of these compounds, but also to oligomeric form, e.g., in which the degree of polymerization is up to about 10, in particular dimers, trimers and tetramers. Mixed oligomers of propanediols, acetoxypropanals, acetoxypropanols, hydroxypropanals are also. possible and are included within the scope of such terms

Suitable vinyl carboxylates are of the formula R—C(═O)—O—CH═CH₂ where R is an organic group constituting the residue of an organic acid, such as formic acid, acetic acid, propionic acid, benzoic acid, etc., with the R ordinarily being an alkyl or aryl group,.or any organic hydrocarbyl radical, for example an alkyl group of 1 to 6 carbon atoms, phenyl, tolyl etc. Vinyl carboxylate with any carboxylate group can be employed in the present process, but ordinarily there is no advantage in utilizing a vinyl ester other than an ester of one of the simple organic acids. The carboxylate portion of the vinyl ester does not constitute part of propanediols in any event, Vinyl acetate is a suitable vinyl carboxylate, which can be prepared by know procedures from acetylene or ethylene and acetic acid. Vinyl acetate was generally used in the present invention and therefore it is used in most of the examples and discussions herein. However, it is to be understood that any of the other vinyl carboxylates can be substituted for vinyl acetate in the examples and elsewhere herein.

The vinyl carboxylate is present particularly at the start of, the hydroformylation step of the present invention in widely varying amounts, for example, at a concentration in the range of about 0.01% to about 95%, preferably about 0.5% to about 75%, by weight based on the total weight of reactants, catalyst and liquid medium present during this step.

The hydroformylation reaction is carried out in a hydroformylation vessel, which can be a pressure reaction vessel such as a bubble column or agitated tank, operated batch wise or in a continuous manner. Separate or combined streams of vinyl acetate, carbon monoxide and hydrogen are charged into the hydroformylation vessel. The feed streams are contacted in the presence of a cobalt catalyst, that is, a cobalt carbonyl, composition. The hydrogen and carbon monoxide will generally be introduced in the reaction vessel in a molar ratio within the range of about 1:2 to about 8:1, preferably about 1. 5:1 to about 5: 1.

The reaction is carried out under conditions effective to produce a hydroformylation reaction product mixture containing a major portion of acetoxypropanals and a minor (as minimum as possible) portion of ethyl acetate and acetic acid. Generally, the hydroformylation reaction is carried out at elevated temperatures of about 140° C., preferably about 90° C. to about 130° C., most preferably about 100° C. to about 120° C., and at a pressure within the range of about 500 to about 5000 psi preferably (for process economics) about 1000 to about 3500 psi. In general, relatively low reaction temperatures (below about 120° C.) and relatively short residence time (about 20 minute to about 1 hr) are preferred. In the practice of the invention, it is possible to achieve acetoxypropanal yields (based on vinyl acetate converted) greater than 80%, with formation of greater than 7% acetoxypropanals, at rates greater than 30 hr⁻¹ (Catalytic rates are referred to heresy in terms of “turn over frequency” or “TOF” and are expressed in units of moles of acetoxypropanals/mole of cobalt/hr). The hydroformylation contacting or step takes place in the presence of, e.g., in, a suitable liquid medium, which is preferably a solvent for the vinyl carboxylate and transition metal containing catalyst composition. Among the suitable liquid media are aliphatic hydrocarbon components including hexane, cyclohexane, decane and the like, aromatic hydrocarbon components, including benzene, toluene, xylenes and the like, ethers, including high molecular weight ethers, polyethers, especially glycol polyethers, and cyclic ethers, amides, sulpholanes, chlorinated solvents such as dichloromethane, dichloroethane, chlorobenzene, dichlorobenzene and the like, alcohols, ketones, esters and mixtures thereof. Specific examples of the suitable liquid medium include toluene, cyclohexane, dichloroethane, dichlorobenzene, and methyl tertiary butyl ether.

An important preferred feature of the present invention is the use of liquid media for hydroformylation of vinyl carboxylates, particularly vinyl carboxylates of simple low molecular weight acids such as those containing two to about five carbon atoms, especially vinyl acetate, in which the products propanediols and/or acetoxypropanals are insoluble or immiscible over a useful range of conditions. In particular the present catalysts have substantial activity and selectivity for propanediols and/or acetoxypropanals in the hydroformylation of low molecular weight vinyl carboxylates using liquid media, which heretofore have been less suited for such hydroformylation service. Such liquid media, in particular hydrocarbons and mixtures thereof, are not only effective in the vinyl carboxylate hydroformylation but also form a two phase mixture with the products propanediols and/or acetoxypropanals, at conditions so that products are separable from the liquid medium, using conventional phase separation techniques, such as centrifugation, decantation and the like.

Thus, in one embodiment of the present invention, propanediols and/or acetoxypropanals production process includes a step or steps in which the reaction mixture, after hydroformylation, in particular the liquid medium and the products acetoxypropanals, are caused to form a liquid medium rich phase and an acetoxypropanal-rich phase. In one particularly useful embodiment, the reaction mixture is cooled from hydroformylation reaction temperature to provide for the recovery of such phases. For example, cooling or maintaining the reaction mixture at a temperature in the range of about −50° C. to about 50° C., can cause the desired phase formation. Care should be taken to avoid temperatures at which a significant amount of the liquid medium solidifies. The liquid medium-rich phase has a higher concentration of the liquid medium and preferably a higher concentration of catalyst components, than that present in the total liquid reaction mixture and products acetoxypropanals after hydroformylation. Analogously, the acetoxypropanal-rich phase has a higher concentration of acetoxypropanals than that present in the total or combined liquid reaction mixture.

The liquid medium-rich phase and the acetoxypropanal-rich phase, which are in contact with each other, are preferably separated, e g., using conventional phase separation techniques, to form a separate acetoxypropanal-rich material. This separated acetoxypropanal-rich material, which includes a minor amount of other materials such as liquid medium and possibly acetoxypropanols and other components in the reaction mixture, is preferably further processed to produce the desired propanediols. The separated acetoxypropanal-rich material can be used directly in the hydrogenation step to produce acetoxypropanols. This direct hydrogenation is particularly useful when the liquid medium included with the acetoxypropanal-rich material is selected from hydrocarbons, such as cyclohexane, xylenes etc. Such liquid medium materials do not substantially detrimentally affect the hydrogenation step.

A yet another important feature of the present invention is a use of an essentially non-water miscible liquid medium for hydroformylation reactions. In general an ideal solvent for modified/non-modified cobalt carbonyl catalyzed hydroformylation of vinyl carboxylates, will solubilize carbon monoxide, will be essentially non water miscible and will exhibit law to moderate polarity such that the acetoxypropanal intermediate will be solubilized to the desired concentration of at least about 5 wt % under hydroformylation conditions, while significant solvent will remain as a separate phase upon water extraction. By essentially non-water-miscible is meant that the solvent has solubility in water at 25 wt % of less than 25 wt %, so as to form a separate hydrocarbon-rich phase upon water extraction of acetoxypropanals from the hydroformylation reaction mixture. Preferably this solubility is. less than 10% and more preferably less than about 3 wt %. The solubilization of carbon monoxide in the selected solvent will generally be greater than about 0. 15v/v (1 atm. 25° C.), preferably greater than 0.25 v/v, as expressed in terms of Ostwald coefficients. The preferred class of solvents may include aromatic/allylic hydrocarbons, alcohols and ethers, halogenated solvents etc. Such solvents include toluene, dichloromethane, dichloroethane, dichlorobenzene, chlorobenzene, diethyl ether, methyl-t-butyl ether, ethyl-t-butyl ether. Blends of solvents such as tetrahydrofuran/toluene, tetrahydrofuran/heptane, and t-butylalcohol/hexane can also be used to achieve the desired solvent properties. The currently preferred solvents because of the high yields of acetoxypropanals which can be achieved under moderate reaction conditions, are toluene, dichloromethane, dichloroethane and methyl-t-butyl ether.

The catalysts for vinyl carboxylate hydroformylation process are transition metal carbonyl catalysts with or without ligands. Group VIII transition metals including rhodium, ruthenium, iridium, cobalt, palladium, nickel etc. are more suitable for this process; still more suitable are cobalt carbonyl and rhodium carbonyl and the mixtures thereof. Transition metal carbonyl to be used as a catalyst for vinyl carboxylate hydroformylation may also be modified by ligands comprised of organic compounds of Group V elements such as triaryl-and trialkyl-phosphines, arsines, amines etc. The catalyst can be supplied to the hydroformylation reactor in essentially any form including metal, supported metal, hydroxide, oxide, carbonate, sulfate, acetylacetonate, salt of a carboxylic acid, or as an aqueous salt solution, for example, it may be supplied directly as a cobalt or rhodium carbonyl such as dicobalt octacarbonyl cobalt hydridocarbonyl, tetrarhodium dodecacarbonyl, dicarbonylacetylacetatonatorhodium etc., or organometallic complexes of the transition metals and ligands such as HRh(CO)(PPh₃)₃, HCo(CO)₃(PBu₃) etc. can be added or operating conditions can be adjusted such that carbonyls are formed in situ via reaction with H₂ and CO, as described in J. Falabe, “Carbon Monoxide in Organic Synthesis” Springer-Veriag, N.Y. (1970). In general, catalyst formation conditions will include a temperature of at. least 50C and a carbon monoxide partial pressure of at least about 100 psi. For more rapid reaction, temperatures of about 120° C. to 200° C., should be employed, at CO pressures of at least about 500 psi. Addition of high surface area activated carbons or zeolites, especially those containing or supporting platinum or palladium metal can accelerate cobalt carbonyl formation from noncarbonyl precursors. The resulting catalyst is maintained under a stabilizing atmosphere of carbon monoxide, which also provides protection against exposure to oxygen.

The amount of transition metal present in the reaction mixture will vary depending upon the metal used and other reaction conditions but will generally fall within the range of about 0.01 to 1 wt %, preferably about 0.05% to about 0.3 wt %, based on the weight of the reaction mixture, in case of cobalt catalyst-and about 0.005 to 0.5 wt %, preferably about 0.01 to 0.1 wt % based on the weight of the reaction mixture, in case of rhodium catalyst.

The hydroformylation reaction with cobalt carbonyl catalyst may include a lipophillic amine promoter to accelerate the rate without imparting hydrophilicity (water solubility) to the active catalyst. By “lipophillic” is meant that most of the amine tends to remain in the organic phase after extraction of acetoxypropanals with water. The amine will be present in an amount effective to promote the hydroformylation reaction to acetoxypropanals, generally an amount within the range of about 0.01 to about 9 moles, preferably within the range of 0.6 to 3 moles based on cobalt.

Suitable lipophillic amines include those represented by formula (3): 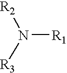
in which each of R₁, R₂, and R₃in independently selected from hydrogen and/or unsubstituted and non-interfering substituted C₁-C₂₁ hydrocarbons. Some suitable primary, secondary and tertiary amines may include dimethyl amine, diethyl amine n-butyl amine, di-n-butyl amine, sec-butyl amine, di-sec-butyl amine, n-hexyl amine, di-n-hexyl amine, octyl amine, di(2-ethylhexyl) amine, dodecyl amine, steryl amine, allyl amine, diallyl amine, crotyl amine, dicrotyl amine, cyclopentyl amine, dicyclopentyl amide, dicyclohexyl amine, benzyl amine, dibenzyl amine, phenyl ethyl amine, diphenyl amine, cinnamyl amine, dicinnamyl amine, aniline, o-, m- and p- toludines, 1,2,3-xylidine, 1,2,4- xylidines, 1,3,5-xylidines, 1,3,4-xylidine, mesidine, pacudocumidine, monoethyl aniline, benzyl aniline, pyrrole, guanidine, triethyl amine, trimethyl amine, tributyl amine, trihexyl amine, triphenyl amine, diphenyl methyl amine and the like. Two or more of the R groups together may form a ring structure, as in Pyridine and substituted pyridines described by formula (4): 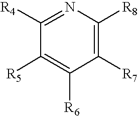
in which each of the R groups is independently selected form hydrogen and C₁-C₂₅ hydrocarbons, two or more may form a cycloaliphatic or aromatic ring, and R₄ and R₈ are not bulky groups such as t-butyl. The promoter is preferably, a non-chelating amide of conjugate acid (pKa about 5-11). Such tertiary amines include dimethyldodecylamine, pyridine, 4-(1-butylpentyl)pyridine, quinoline, isoquinolein, lipdine and quinaldine. The preferred amine, because of its availability and demonstrated promotion of vinyl carboxylate hydroformylation, is pyridine.

The rhodium catalyzed hydroformylation of vinyl carboxylate may include a phosphorous, nitrogen, arsine or stilbine containing ligands such as for example monodented ligands such as triphenyl phosphine, tributyl phosphine, triphenyl amine, triphenyl arsine, triethyl amine, tricyclohexyl amine, pyridine, substituted pyridines such as 4-methyl pyridine, etc or polydented ligands such as diphenyl phosphinoethane, diphenyl phosphinopropane, diphenyl phosphinobutane, diphenyl phosphinopentane, bipyridine, terpyridine etc.

It has also been found that good yields of optically active 2-acetoxypropanal can be achieved in presence of the optically active ligand. For instance, the catalyst for vinyl carboxylate hydroformylation to optically active 2-acetoxypropanal, can be prepared by dissolving a soluble metal compound in a suitable solvent together with a ligand wherein the ratio of the ligand is at least one equivalent of ligand per mole of metal. Likewise, it has been found that the catalyst can be formed in situ by adding a soluble metal compound to the reaction mass together with the proper amount of optically active ligand either before or during hydroformylation. The optically active 2-acetoxypropanal can then be converted to optically active 2-acetoxypropanol by hydrogenation or 2-hydroxy propanal by hydrolysis or optically active 1,2-propanediol. Either d- or l- form of 2-acetoxypropanol, 2-hydroxypropanal and 1,2-propanediol can be prepared by appropriate selection of the ligands for the catalyst in the hydroformylation reaction.

For the cobalt catalyzed hydroformylation of vinyl acetate monomer, it is generally preferred to regulate the concentration of water in the hydroformylation reaction mixture, as excess amount of water may reduce acetoxypropanal selectivity below acceptable levels, may induce formation of a second liquid phase and may convert the catalyst in an inactive form. At low concentrations, water can assist in promoting the formation of the desired cobalt carbonyl catalyst species. Acceptable water levels will depend upon the solvent used, with more polar solvents are generally more tolerant to higher water concentrations. For example, optimum water levels for hydroformylation in methyl-t-butyl-ether solvent are believed to be within the range of about 1 to about 2.5 wt %.

Following the hydroformylation reaction, hydroformylation reaction product mixture containing acetoxypropanals, the reaction solvent, acetoxypropanols in minor amounts, the transition metal catalyst, ligands (if used) and a minor amount of reaction by-products such as ethyl acetate, acetic acid, is passed to the extraction vessel, wherein an aqueous liquid, generally water and optional mobilizing solvent, are added for extraction and concentration of acetoxypropanals for the subsequent hydrogenation step. Liquid extraction can be effected by any suitable means, such as mixer-settlers, packed or frayed extraction columns, or rotating disk contactors. Extraction can, if desired, be carried out in multiple stages. The water containing hydroformylation product mixture can optionally be passed to a settling tank for resolution of the mixture into aqueous and organic phases. The amount of water added to the hydroformylation product mixture will generally be such as to provide a water: mixture ratio within the range of about 1:1 to about 1:20, preferably about 1;5 to about 1:15. The addition of water at this stage may have the additional advantage of suppressing formation of the undesired heavy ends. Extraction with relatively small amount of water provides an aqueous phase with greater than 20 wt % acetoxypropanals, preferably greater than 25 wt % acetoxypropanals, permitting economical hydrogenation of acetoxypropanals to acetoxypropanols. The water extraction is preferably carried out at a temperature within the range of about 25° C. to 55° C., with higher temperatures avoided to minimize condensation products (heavy ends) and catalyst disproportionation to inactive, water-soluble cobalt species. In order to maximize the catalyst recovery, it is optional but preferred to perform the water extraction under 50 to 300 psi carbon monoxide.

Yet another feature of the present invention is a use of reverse biphasic system for efficient hydroformylation in continuous mode. In some cases of hydroformylation of vinyl acetate, water can be added as a co-solvent so as to create a separate phase and thus exaction of the hydroformylation products can be performed during the hydroformylation step. An advantage of such reverse biphasic system is easy recycle of the catalyst with the recycling of the organic phase in continuous operation. Such reverse biphasic systems are more suitable if rhodium catalyst is used for hydroformylation.

Commercial operation will require efficient transition metal catalyst with essentially complete recycle of the transition metal to the hydroformylation reaction. The preferred catalyst recovery process involves two steps, beginning with above described water extraction of acetoxypropanals under carbon monoxide from the hydroformylation product mixture. Majority of the transition metal catalysts remain in organic solvent phase with remaining catalyst passing into water phase. Organic phase can be recycled to hydroformylation reactor with optional purge of heavy ends. Optional further removal of catalyst in the water layer can be affected by suitable method such as complete or partial oxidation of metal followed by precipitation and filtration, distillation, deposition on solid support or extraction using a suitable extractant preferably prior to final metal removal by ion exchange.

The organic phase containing the reaction solvent and a major portion of the catalyst can be recycled from the extraction vessel to the hydroformylation reactor. The aqueous product mixture is passed through hydrogenation vessel and reacted with hydrogen in the presence of hydrogenation catalyst to produce hydrogenation product mixture containing acetoxypropanols. The hydrogenation step may also revert some heavy ends to acetoxypropanols.

Hydrogenation of the acetoxypropanals to acetoxypropanols can be carried out in aqueous solution at an elevated temperature of at least about 40° C., generally within the range of about 50° C. to about 175° C. under a hydrogen pressure of at least about 100 psi generally within the range of about 200 to about 2000 psi. The reaction is carried out in the presence of a hydrogenation catalyst such as any of those based upon group VIII metals including Nil Co, Ru, Pt and Pd as well as Cu, Zn, Cr, Au catalysts including bulk supported and fixed bed forms provide acceptable activities and selectivities at moderate costs. Highest yields are achieved under slightly acidic reaction conditions.

It has been found that hydrogenation catalysts are deactivated more rapidly than desired in the preparation of acetoxypropanols via the hydrogenation of acetoxypropanals. The deactivation of the catalyst is believed to be due to the adverse interaction between impurities in the aqueous acetoxypropanal feed and the hydrogenation catalysts employed.

It has been found that treatment of the aqueous acetoxypropanal feed with a purifying agent prior to hydrogenation improves performance and lifetime of the hydrogenation catalysts. Initial activity of the hydrogenation catalysts is particularly enhanced and it has been found that the catalyst recycles are very effective in case of a batch reactors if the feed is treated with a purifying agent prior to hydrogenation. Purifying agents useful in the process of the present invention comprise purifying carbons, purifying silica compositions, diatomaceous earth and zeolites.

The hydrogenation is carried out by methods known in the art in known reactors, the liquid reaction mixture, the catalyst and hydrogen being thoroughly contacted with one another. In case of suspension hydrogenation, stirred reactors or flow reactors are particularly suitable. The suspension hydrogenation is carried out at a temperature of preferably 40° C. to 60° C. to an acetoxypropanal conversion of 50 to 80% and is then continued, preferably at 110° C. to 150° C. to an acetoxypropanal conversion of substantially 100%. Acetoxypropanol containing stream, obtained after the hydrogenation step is passed to the hydrolysis reactor for further hydrolysis to propanediols.

The hydrolysis of the acetoxypropanols to propanediols can be accomplished in the presence of any of the usual acid or base catalysts for ester hydrolysis. Thus, mineral acids, sulphonic acids, ion exchange resins, many types of solid acids such as ZSM-5 etc., heteropoly acids, carboxylic acids, sodium hydroxide, potassium hydroxide etc. can be used.

The hydrolysis reaction is performed in the same aqueous mixture in different types of the reactors such as slurry batch reactors, trickle bed reactors, packed column reactors and those known in the art of reaction engineering. The temperature of the hydrolysis reaction can vary depending upon the catalyst used and the type of the reactor. Generally, for an ion exchange catalyzed hydrolysis reaction the temperature of the reaction can be from room temperature to 120° C., preferably from 50° C. to 80° C.

In the present invention, ‘Amberlite IR-120’ cation exchange resin is generally used for the hydrolysis of a mixture of 2- and 3-acetoxypropanols to 1,2- and 1,3-propanediols respectively but it is to be understood that other suitable acidic and basic resins can very well be used in place of Amberlite IR-120 resin and hydrolysis can be performed. Mineral acids and bases are also used for hydrolysis, but to avoid the unwanted side reactions and separation problems such acids are used in dilute form, generally in the concentration range of 0.1 to 3 M, preferably in the range of 0.5 to 2 M concentration range. Hydrolysis with mineral acids and bases can take place even at room temperature but a temperature in the range of 40° C. to 100° C. is preferred.

In a process of producing propanediols from vinyl acetate monomer, the reaction sequence can be either hydroformylation to get acetoxypropanals followed by hydrogenation to get acetoxypropanols followed by hydrolysis to get propanediols or hydroformylation to get acetoxypropanals followed by hydrolysis to get hydroxypropanals followed by hydrogenation to get propanediols. However, in the second sequence the acetic acid generated in the hydrolysis reaction should preferably be removed before the hydrogenation step so as to avoid the undesired side reactions. The first sequence of the reaction does not require any separation of the products until a mixture of propanediols and acetic acid is. obtained. Because of considerable differences in the boiling points, acetic acid, 1,2-propanediol and 1,3-propanediol can be separated easily by conventional distillation method.

The present invention process permits the selective and economic synthesis of propanediols at moderate temperatures and pressures. The process involves preparation of reaction product mixture containing acetoxypropanals, then separation of this acetoxypropanals and catalyst by water extraction followed by hydrogenation of the acetoxypropanals to acetoxypropanols and followed by hydrolysis of acetoxypropanols to PDOs.

### EXAMPLES

The following examples are given be way of illustration and therefore should not be construed to limit the scope of the present invention.

### EXAMPLE 1

A 50 ml stirred batch reactor was charged under nitrogen with 0.1025 g dicobalt octacarbonyl, 17.1805 g toluene, and 4.48 g vinyl acetate monomer (VAM). The reactor was flushed twice with 1:1 mixture of carbon monoxide and hydrogen and was filled with 1400 psi of 1:1 CO/H₂. Reactor contents were heated to 120° C. and the reaction was run for 110 minutes at 1200 rpm stirring speed. Syn gas pressure of 1400 psi was maintained by refilling of the gas after every 50 psi absorption. After 110 minutes, the reactor was cooled to 20° C. and the pressure was vent off. Final sample was analyzed on GC (with flame ionization detector). The analysis showed 90% of the VAM was converted with 95% selectivity towards acetoxypropanals and ethyl acetate and acetic acid in traces. The ratio of 2-acetoxypropanal to 3-acetoxypropanal was 55:45.

8 ml of deionised, degassed water was added to the above charge and the reactor was again pressurized to 300 psi carbon monoxide and stirred at 1200 rpm for 10 minutes. The contents were allowed to stand for 10 minutes and the aqueous phase was removed under nitrogen atmosphere after venting off the carbon monoxide. The organic phase retained it's wine-red color and the aqueous phase was colorless. Such extraction was repeated twice with 8 ml deionised, degassed water. The aqueous phase was combined and analyzed. It was found that 92% of the acetoxypropanals were extracted in the aqueous phase. The combined aqueous phase was added to a 50 ml stirred batch reactor. 100 mg of 3% RU/Al₂O₃catalyst was added to the aqueous phase, the reactor was flushed twice with hydrogen gas and pressurized to 1000 psi with hydrogen. It was heated to 50° C. and the reaction was run for 3 hours at 1200 rpm stirring speed. The final sample was analyzed with GC and 90% of the acetoxypropanals were converted into acetoxypropanols.

The catalyst 3% Ru/Al₂0₃, was filtered off and the aqueous solution containing acetoxypropanols was transferred again to the 50 ml reactor. 100 mg Amberlite IR 120 resin was added to the reactor, it was heated to 80° C., and was stirred at 1200 rpm for 3 hours. The GC analysis of the final sample showed 85% conversion of acetoxypropanols with 97% selectivity to propanediols. 1,2-propanediol and 1,3- propanediol were in 58:42 ratio.

### EXAMPLE2

The organic phase obtained after extraction of products in example 1 was charged as it is in a 50 ml reactor, 2.954 g VAM was added to this organic phase. The reactor was flushed twice with 1:1 mixture of carbon monoxide and hydrogen and was filled with 1500 psi of 1:1 CO/H₂. Reactor contents were heated to 120° C. and the reaction was run for 120 minutes at 1200 rpm stirring speed. Syn gas pressure of 1500 psi was maintained by refilling of the gas after every 50 psi absorption. The reactor was cooled after 120 minutes, to 20° C. and the, pressure was vent off -The GC analysis of the final sample showed 90% VAM converted to acetoxypropanals with n/iso ratio 42:58. 4.6% acetic acid and 3% ethyl acetate was formed.

8 ml of deionised, degassed water was added to the above charge and the reactor was again pressurized to 300 psi carbon monoxide and stirred at 1200 rpm for 10 minutes. The contents were allowed to stand for 10 minutes and the aqueous phase was removed under nitrogen atmosphere after venting off the carbon monoxide. The organic phase retained its wine-red color and the aqueous phase was colorless. Such extraction was repeated twice with 8 ml deionised, degassed water. The aqueous phase was combined and analyzed. It was found that 93% of the acetoxypropanals were extracted in the aqueous phase. The combined aqueous phase was passed through an activated carbon column for removal of impurities and added to a 50 ml stirred batch reactor. 100 mg of 3% Ru/Al₂0₃ catalyst was added to the aqueous phase, the reactor was flushed twice with hydrogen gas and pressurized to 1000 psi with hydrogen. It was heated to 50° C. and the reaction was run for 3 hours at 1200 rpm stirring speed. The final sample was analyzed with GC and 92% of the acetoxypropanals were converted into acetoxypropanols.

### EXAMPLE 3

A 50 ml stirred batch reactor was charged under nitrogen with 200 mg cobalt acetate, and 21 g toluene. The reactor was flushed twice with 1:1 mixture of carbon monoxide and hydrogen and was filled with 1000 psi of 1:1 CO/H₂. Reactor contents were heated to 200° C. and the syn gas pressure was increased up to 2000 psi, the reaction was run for 45 minutes at 1200 rpm stirring speed, the reactor was cooled to 120° C. and 2.653 g VAM was added with high pressure addition device. The reaction was run at 120° C., 1800 psi 1:1 CO:H₂ and 1200 rpm stirring speed for 60 minutes. Syn gas pressure of 1800 psi was maintained by refilling of the gas after every 50 psi absorption The reactor was cooled to 20° C. after 60 minutes and the pressure was vent off. GC analysis of the final sample showed 85% conversion of VAM with 92% selectivity towards acetoxypropanals, 3% acetic acid and 2% ethyl acetate were detected, The ratio of 2-acetoxypropanal to 3-acetoxypropanal was 46:54

### EXAMPLE 4

A 50 ml stirred batch reactor was charged under nitrogen with 0.1 g dicobalt octacarbonyl, 28.197 g dichloromethane and 3.135 g VAM. The reactor was flushed twice with 1:1 mixture of carbon monoxide and hydrogen and was filled with 1500 psi of 1:1 C0/H₂. Reactor contents were heated to 120° C. and the reaction was run for 60 minutes at 1200 rpm stirring speed. Syn gas pressure of 1500 psi was maintained by refilling of the gas after every 50 psi absorption. The reactor was cooled to 20° C. after 60 minutes and the pressure was vent off. GC analysis of the final sample showed VAM conversion of 85% with acetoxypropanals selectivity 94%. Traces of acetic acid and ethyl acetate were found. The ratio of 2-acetoxypropanal to 3-acetoxypropanal was 42:58. Thus, with dichloromethane as a solvent the selectivity towards the normal aldehyde was found to increase substantially.

### EXAMPLE 5

A 50 ml stirred batch reactor was charged under nitrogen with 0.105 g dicobalt octacarbonyl, 28.275 g 1,2-dichlorobenzene and 3.1030 g VAM. The reactor was flushed twice with 1:1 mixture of carbon monoxide and hydrogen and was filled with 1500 psi of 1:1 CO/H₂. Reactor contents were heated to 120° C. and the reaction was run for 60 minutes at 1200 rpm stirring speed. Syn gas pressure of 1500 psi was maintained by refilling of the gas after every 50 psi absorption. After the reaction, reactor was cooled to 20° C. and the pressure was vent off. GC analysis of the final sample showed VAM conversion of 88% with acetoxypropanals selectivity 93%. Traces of acetic acid and ethyl acetate were found. The ratio of 2-acetoxypropanal to 3-acetoxypropanal was 43:57. Thus, with 1,2-dichlorobenzene as a solvent the selectivity towards the normal aldehyde was found to increase substantially.

8 ml of deionised, degassed water was added to the above charge and the reactor was again pressurized to 300 psi carbon monoxide and stirred at 1200 rpm for 10 minutes. The contents were allowed to stand for 10 minutes the aqueous phase was removed under nitrogen atmosphere after venting off the carbon monoxide. The organic phase retained its wine-red color and the aqueous phase was colorless. Such extraction was repeated twice with 8 ml deionised, degassed water. The aqueous phase was combined and analyzed. It was found that 90% of the acetoxypropanals were extracted in the aqueous phase. The combined aqueous phase was added to a 50 ml stirred batch reactor. 100 mg of Raney Ni catalyst was added to the aqueous phase, the reactor was flushed twice with hydrogen gas and pressurized to 1000 psi with hydrogen. It was heated to 50° C. and the reaction was rpm for 3 hours at 1200 rpm stirring speed. The final sample was analyzed with GC, which showed 75% of the acetoxypropanals were converted into acetoxypropanols.

The catalyst Raney Ni, was filtered off and the aqueous solution containing acetoxypropanols was transferred again to the 50 ml reactor. 100 mg Amberlite IR 120 resin was added to the reactor, it was heated to 80° C., and was stirred at 1200 rpm for 3 hours. The GC analysis of the final sample showed 81% conversion of acetoxypropanols with 97% selectivity to propanediols. 1,2-propanediol and 1,3-propanediol were in 45; 55 ratio.

### EXAMPLE 6

A 50 ml stirred batch reactor was charged under nitrogen with 0.051 g dicobalt octacarbonyl, 0.0575 g pyridine, 16.2985 g toluene and 5.4335 g VAM. The reactor was flushed twice with 1:1 mixture of carbon monoxide and hydrogen and was filled with 600 psi of 1:1 CO/H₂. Reactor contents were heated to 120° C. and the reaction was run for 60 minutes at 1200 rpm stirring speed Syn gas pressure of 600 psi was maintained by refilling of the gas after every 50 psi absorption. After 60 minutes, the reactor was cooled to 20° C. and the pressure was vent off. Final sample was analyzed on GC, 92% conversion of VAM with 95% selectivity towards acetoxypropanals was obtained. The ratio of 2-acetoxypropanal to 3-acetoxypropanal was 58:42.

10 ml of deionised, degassed water was added to the above charge and the reactor was again pressurized to 300 psi carbon monoxide and stirred at 1200 rpm for 10 minutes. The contents were allowed to stand for 10 minutes. The aqueous phase was removed under nitrogen atmosphere after venting off carbon monoxide. The organic phase retained its wine. red color and the aqueous phase was colorless. Extraction was repeated twice with 8 ml deionised degassed water. Analysis of combined aqueous phase showed that 90% of acetoxypropanals were extracted in the aqueous phase. The combined aqueous phase was added to a 50 ml stirred batch reactor. 0.1 g Amberlite IR 120 resin was added to the aqueous phase and reactor was heated to 80° C. Reaction was run at 1200 rpm and 80° C. for 3 hours. Reactor was cooled to 20° C. and final sample was analyzed on GC. 82% of acetoxypropanals were converted to hydroxypropanals and acetic acid. The ratio of 2-hydroxypropanal to 3-hydroxypropanal was 60:40, Thus, some 3-acetoxypropanal was decomposed.

### EXAMPLE 7

A 50 ml stirred batch reactor was charged under nitrogen with 0.049 g dicobalt octacarbonyl, 0.085 g 4-acetyl pyridine, 18.82 g toluene and 2.6355 g VAM. The reactor was flushed twice with 1.1 mixture of carbon monoxide and hydrogen and was filled with 600 psi of 1:1 COH₂. Reactor contents were heated to 120° C. and the reaction was run for 100 minutes at 1200 rpm stirring speed. Syn gas pressure of 600 psi was maintained by refilling of the gas after every 50 psi absorption. After 100 minutes the reactor was cooled to 20° C. and the pressure was vent off. Analysis of the final mixture showed 90% VAM conversion with 95% towards acetoxypropanals. The n/iso ratio of the acetoxypropanals was 42:58.

8 ml of deionised, degassed water was added to the above charge and the reactor was again pressurized to 200 psi carbon monoxide and stirred at 1200 rpm for 10 minutes. The contents were allowed to stand for 10 minutes the aqueous phase was removed under nitrogen atmosphere after venting off the carbon monoxide. The organic phase retained it's wine-red color and the aqueous phase was colorless. Such extraction was repeated twice with 8 ml deionised, degassed water. The aqueous phase was combined and analyzed. It was found that 95% of the acetoxypropanals were extracted in the aqueous phase. The combined aqueous phase was added to a 50 ml stirred batch reactor. 100 mg of 3% Ru/Al₂O₃ catalyst was added to the aqueous phase, the reactor was flushed twice with hydrogen gas and pressurized to 1000 psi with hydrogen. It was heated to 50° C. and the reaction was run for 3 hours at 1200 rpm stirring speed. The final sample was analyzed with GC and 87% of the acetoxypropanals were converted into acetoxypropanols.

The catalyst 3% Ru/Al₂O₃, was filtered off and the aqueous solution containing acetoxypropanols was transferred again to the 50 ml reactor. 10 ml of 1 M HCI was added to the reactor, it was heated to 50° C., and was stirred at 1200 rpm for 30 minutes. The GC analysis of the final sample showed 85% conversion of acetoxypropanols. with corresponding amount of acetic acid and 97% selectivity to propanediols. 1,2-propanediol and 1,3-propanediol were in 60:40 ratio.

### EXAMPLE 8

A 50 ml stirred batch reactor was charged under nitrogen with 0.015 mg HRh(CO)(PPh₃)₃, 11.223 g cyclohexane and 9.49 g VAM . The reactor was flushed twice with 1:1 mixture of carbon monoxide and hydrogen and was filled with 900 psi of 1:1 CO/H₂. Reactor contents were heated to 120° C. and the reaction was run for 120 minutes at 1200 rpm stirring speed. Syn gas pressure of 900 psi was maintained by refilling of the gas after every 50 psi absorption. After 120 minutes, the reactor was cooled to 20° C. and the pressure was vent off. Two distinct layers were formed. GC analysis of the phase showed one phase with nearly 92% acetoxypropanals. Overall conversion of vinyl acetate monomer was found to be 85%. 7% acetic acid was formed. The % selectivity for acetoxypropanals was 92%. The ratio of the 2-acetoxypropanal to 3-acetoxypropanal was 90 10.

### EXAMPLE 9

A 50 ml stirred batch reactor was charged under nitrogen with 0.0225 mg HRh(CO)(PPh₃)₃, 0.02 g triphenyl phosphine, 10.035 g toluene, 11.38 g water and 2.858 g VAM. Reactor was flushed twice with 1:1 mixture of carbon monoxide and hydrogen and was filled with 800 psi of 1:1 CO/H₂. Reactor contents were heated to 80° C. and reaction was run for 120 minutes at 1200 rpm stirring speed. Syn gas pressure of 800 psi was maintained by refilling of gas after every 50 psi absorption. After 120 minutes, reactor was cooled to 20° C. and the pressure was vent off. GC analysis of final samples of organic phase and aqueous phase showed 95% VAM conversion with 98% selectivity towards acetoxypropanals. 92% acetoxypropanals were found in the aqueous phase and only 8% remained in organic phase. The aqueous phase was totally colorless whereas organic phase retained its saffron colors. The ratio of the 2-acetoxypropanal to 3-acetoxypropanal was 9:1.

The organic phase of the above experiment was again charged under nitrogen atmosphere into the reactor with 12 g water and 2.74 g VAM. The reactor was flushed twice with 1:1 mixture of CO:H2 and was pressurized to 800 psi with 1:1 CO/H₂. The reactor was heated to 80° C. and reaction was carried out at 1200 rpm for 105 minutes. The reactor was cooled to 20° C. and the pressure was vent off. GC analysis of final samples of organic phase and aqueous phase showed 96% VAM conversion with 98% selectivity towards acetoxypropanals. 90% acetoxypropanals were found in the aqueous phase and only 10% remained in the organic phase. The aqueous phase was totally colorless whereas organic phase retained its saffron colors. The ratio of the 2-acetoxypropanal to 3-acetoxypropanal was 9:1. Thus, the costly rhodium catalyst was recycled efficiently with such type of reverse biphasic systems.

### EXAMPLE 10

The aqueous phase of the example 9 was taken in a hydrogenation reactor. 100 mg of 3% Ru/Al₂O₃ catalyst was added to the aqueous phase, the reactor was flushed twice with hydrogen gas and pressurized to 1000 psi with hydrogen. It was heated to 50° C. and the reaction was run for 1.5 hours at 1200 rpm stirring speed. Temperature of the reactor was increased to 150° C. and the reaction was run for 30 minutes at 1200 rpm. Reactor was cooled and the pressure was vent off. The final sample was analyzed with GC and 90% of the 2-acetoxypropanal was converted into 2-acetoxypropanol whereas 3-acetoxypropanol was found only in trace amounts.

The Main Advantages of the Present Invention are:

1) Propanediols are prepared from a non-toxic, non-corrosive, cheap and readily available substrates such as vinyl carboxylates
2) Convenient catalyst-product separation and all the catalysts used for the three steps are recyclable
3) Carboxylic acid generated in the final step can be easily recovered and recycled to prepare vinyl carboxylates
4) Less severe conditions of temperature and pressure are used for all the three steps involved
5) The process can be used to prepare many useful intermediates such as 2- and 3-acetoxypropanols, 2- and 3-acetoxypropanols, 2- and 3-hydroxypropanals.
6) High yields of the desired products and minimum side reactions in all the three steps of the process.

1. A process for preparing dioxy-functionalized propane compounds of formula X—CH₂—CH(Y)—CH₂-Z wherein X=R—C(═O)—O— or H or —OH, Y and Z=—H or —OH or —C(═O)H, and wherein at a time only one of X, Y and Z is —H which comprises:
(a) contacting a vinyl carboxylate with carbon monoxide and hydrogen in a solvent in the presence of transition metal catalyst, to obtain an intermediate product mixture comprising of 3-carboxypropanal and 2-carboxypropanal;
(b) adding water to the intermediate product mixture of 3-carboxypropanal and 2-carboxypropanal to extract the carboxypropanals into water to obtain an aqueous phase comprising of carboxypropanals and an organic phase comprising of transition metal catalyst and separating the aqueous phase from the organic phase;
(c) contacting the aqueous phase comprising of carboxypropanals with hydrogen in presence of a heterogeneous hydrogenation catalyst to obtain a hydrogenation product mixture comprising of 3-carboxypropanol and 2-carboxypropanol in aqueous phase and separating the hydrogenation catalyst from the aqueous phase;
(d) contacting the aqueous phase obtained in (c) comprising of carboxypropanols with a hydrolysis catalyst at a temperature within the range of 30° C. to 120° C. at least during a portion of a hydrolysis step to provide a product mixture comprising of 1,3- and 1,2-propanediol and a corresponding carboxylic acid;
(e) separating and recovering carboxylic acid, 1,3- and 1,2-propanediols from the aqueous mixture by distillative separation.

2. A process as claimed in claim 1 wherein the organic phase obtained in step (b) is recycled to step (a).

3. A process as claimed in claim 1 wherein step (a) above is carried out in the presence of a promoter or a ligand and at a temperature in the range of 50° C. to 140° C. and a pressure in the range of 500 psi to 5000 psi.

4. A process as claimed in claim 1 wherein step (b) is carried out at a temperature of not more than 100° C.

5. A process as claimed in claim 1 wherein the aqueous phase of carboxypropanals is separated from the organic phase comprising transition metal by phase separation.

6. A process as claimed in claim 1 wherein step (c) is carried out at a minimum temperature of 40° C. and at least for a period of 30 min, while optionally increasing the reaction temperature to 80° C. and hydrogen pressure of at least 200 psi.

7. A process as claimed in claim 1 wherein the aqueous phase comprising carboxypropanols in step (c) is separated from the hydrogenation catalyst by filtration, decantation or centrifugation.

8. A process as claimed in claim 1 wherein the transition metal catalyst used in step (a) comprises a Group VIII transition metal catalyst selected from the group consisting of rhodium, ruthenium, iridium, cobalt, palladium and nickel.

9. A process as claimed in claim 1 wherein the transition metal catalyst is selected from the group consisting of cobalt carbonyl, rhodium carbonyl and any mixture thereof.

10. A process as claimed in claim 1 wherein the transition metal catalyst is modified by a ligand comprising an organic compound of a Group V element selected from the group consisting of triaryl-phosphines, trialkyl-phosphines, arsines and amines.

11. A process as claimed in claim 1 wherein the catalyst of step (a) is used in the form of metal, supported metal, hydroxide, oxide, carbonate, sulfate, acetylacetonate, salt of a carboxylic acid, and an aqueous salt solution.

12. A process as claimed in claim 1 wherein the catalyst of step (a) is used directly in the form of cobalt carbonyl or rhodium carbonyl selected from the group consisting of dicobalt octacarbonyl, cobalt hydridocarbonyl, tetrarhodium dodecacarbonyl, and dicarbonylacetylacetatonatorhodium.

13. A process as claimed in claim 1 wherein the catalyst of step (a) is used in the form of an organometallic complex of the transition metals and ligands selected from the group consisting of HRh(CO)(PPh₃)₃ and HCo(CO)₃(PBu₃).

14. A process as claimed in claim 1 wherein the catalyst of step (a) in carbonyl form is formed in situ through the reaction of the metal with H₂ and CO, and at a temperature of at least 50° C. and a carbon monoxide partial pressure of at least about 100 psi.

15. A process as claimed in claim 1 wherein the temperature for in situ catalyst formation is in the range of 120° C. to 200° C., and the CO pressures is at least about 500 psi.

16. A process as claimed in claim 1 wherein high surface area activated carbon or zeolite containing or supporting platinum or palladium metal are added to the reactor to form cobalt carbonyl from noncarbonyl precursors.

17. A process as claimed in claim 1 wherein the amount of transition metal present in the reaction mixture is in the range of 0.01 to 1 wt % based on weight of reaction mixture.

18. A process as claimed in claim 17 wherein the amount of transition metal present in the reaction m e is in the range of 0.05 to 0.3 wt %, based on weight of reaction mixture.

19. A process as claimed in claim 1 wherein the cobalt catalyst is present in the range of 0.005 to 0.5 wt % based on the weight of the reaction mixture.

20. A process as claimed in claim 19 wherein the cobalt catalyst is present in the range of 0.01 to 0.1 wt % based on the weight of the reaction mixture.

21. A process as claimed in claim 1 wherein the hydroformylation catalyst in step (a) comprises a cobalt complex selected from the group consisting of cobalt acetate, cobalt hydroxide, cobalt oxide, cobalt carbonate, cobalt sulfate, cobalt acetylacetonate, cobalt carboxylate, dicobalt octacarbonyl cobalt hydridocarbonyl and HCo(CO)₃(PBu₃).

22. A process as claimed in claim 1 wherein the hydroformylation catalyst in step (a) comprises a rhodium complex selected from the group consisting of rhodium acetate, rhodium hydroxide, rhodium oxide, rhodium carbonate, rhodium sulfate, rhodium acetylacetonate, rhodium carboxylate, tetrarhodium dodecacarbonyl, dicarbonylacetylacetatonatorhodium and an organometallic complexes of the transition metals and a ligand comprising HRh(CO)(PPh)₃.

23. A process as claimed in claim 21 wherein the cobalt carbonyl catalyst includes a lipophillic ate promoter in an amount effective to promote the hydroformylation reaction to acetoxypropanals.

24. A process as claimed in claim 21 wherein the lipophillic amine promoter is present in an amount m the range of 0.01 to 9 moles based on cobalt.

25. A process as claimed in claim 24 wherein the lipophillic amine promoter is present in an amount in the range of 0.6 to 3 moles based on cobalt.

26. A process as claimed in claim 24 wherein the lipophillic amine is of the formula: 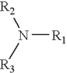
wherein each of R₁, R₂ and R₃ are independently selected from hydrogen and/or unsubstituted and non-interfering substituted C₁-C₂₅ hydrocarbons.

27. A process as claimed in claim 24 wherein the amine is selected from the group consisting of dimethyl amine, diethyl amine, n-butyl amine, di-n-butyl amine, sec-butyl amine, di-sec-butyl amine, n-hexyl amine, di-n-hexyl amine, octyl amine, di(2-ethylhexyl) amine, dodecyl amine, steryl amine, allyl amine, diallyl amine, crotyl amine, dicrotyl amine, cyclopentyl amine, dicyclopentyl amine, dicyclohexyl amine, benzyl amine, dibenzyl amine, phenyl ethyl amine, diphenyl amine, cinnamyl amine, dicinnamyl amine, aniline, o-, m- and p- toludines, 1,2,3-xylidine, 1,2,4- xylidines, 1,3,5-xylidines, 1,3,4-xylidine, mesidine, pacudocumidine, monoethyl aniline, benzyl aniline, pyrrole, guanidine, triethyl amine, trimethyl amine, tributyl amine, trihexyl amine, triphenyl amine and diphenyl methyl amine.

28. A process as claimed in claim 24 wherein In another embodiment of the invention, two of R₁, R₂, and R₃ form a ring structure such as Pyridine and substituted pyridines of the formula 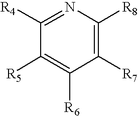
wherein each of R₄, R₅, R₆ and R₇ is dependently selected from hydrogen and C₁-C₂₅hydrocarbons.

29. A process as claimed in claim 24 wherein two of R₁, R₂, and R₃form a ring structure such as Pyridine and substituted pyridines of tie formula 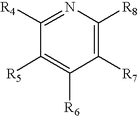
wherein each of R₄, R₅, R₆, and R₇ is independently selected from hydrogen and C₁-C₂₅hydrocarbons.

30. A process as claimed in claim 24 wherein the promoter is a non-chelating amine of a conjugate acid (pKa about 5-11) selected from the group consisting of tertiary amines including dimethyldodecylamine, pyridine, 4-(1-butylpentyl)pyridine, quinoline, isoquinolein, lipdine and quinaldine.

31. A process as claimed in claim 30 wherein the promoter is pyridine.

32. A process as claimed in claim 24 wherein the vinyl carboxylate is of the formula R—C(═O)—O—CH═CH₂ where R is an organic group selected from the group consisting of an alkyl group, aryl group, an organic hydrocarbyl radical comprising an alkyl group with 1 to 6 carbon atoms, phenyl and tolyl.

33. A process as claimed in claim 24 wherein the vinyl carboxylate is a residue of an organic acid selected from the group consisting of formic acid, acetic acid, propionic acid and benzoic acid.

34. A process as claimed in claim 24 wherein the vinyl carboxylate is vinyl acetate.

35. A process as claimed in claim 24 wherein the vinyl carboxylate is present at the start of the hydroformylation step in a concentration in the range of 0.01% to 95% by weight based on the total weight of reactants, catalyst and liquid medium present.

36. A process as claimed in claim 24 wherein the vinyl carboxylate is present at the start of the hydroformylation step in a concentration in the range of 0.5% to 75%, by weight based on the total weight of reactants, catalyst and liquid medium present.

37. A process as claimed in claim 24 wherein the carboxylic acid, corresponding to the vinyl carboxylate used, recovered in step (e) is recycled to produce the vinyl carboxylate starting material.

38. A process as claimed in claim 24 wherein step (a) is carried out with a cobalt/rhodium catalyst and a ligand comprising a nitrogen containing ligand selected from the group consisting of triphenyl amine, triethyl amine, tricyclohexyl amine, pyridine and substituted pyridine.

39. A process as claimed in claim 38 wherein the ligand is 4-methyl pyridine.

40. A process as claimed in claim 24 wherein the ligand used with the cobalt/rhodium catalyst in step (a) comprises a phosphorous, arsine or stilbine containing ligand selected from the group consisting of triphenyl phosphine, tributyl phosphine, triphenyl arsine and tryphenyl stilbine.

41. A process as claimed in claim 24 wherein the ligand used with the cobalt/rhodium catalyst in step (a) comprises a polydentate ligand selected from the group consisting of diphenyl phosphinoethane, diphenyl phosphinopropane, diphenyl phosphinobutane, diphenyl phosphinopentane, bipyridine and terpyridine.

42. A process as claimed in claim 24 wherein the cobalt/rhodium catalyst used in step (a) is used along with a promoter comprising a quaternary ammonium salt selected from the e group consisting of tetra alkyl ammonium halide and tetra alkyl ammonium hydroxide.

43. A process as claimed in claim 24 wherein the promoter used in step (a) along with cobalt/rhodium catalyst comprises a quaternary phosphonium salt selected from the group consisting of tetra alkyl phosphonium halide and tetra alkyl phosphonium hydroxide.

44. A process as claimed in claim 24 wherein the ligand used in step (a) along with cobalt/rhodium catalyst comprises an optically active ligand in order to obtain optically active-chiral 2-acetoxy propanal.

45. A process as claimed in claim 24 wherein the solvent used in step (a) comprises an aliphatic hydrocarbon selected from the group consisting of hexane, cyclohexane and decane.

46. A process as claimed in claim 24 wherein the solvent of step (a) comprises an aromatic hydrocarbon selected from the group consisting of benzene, toluene and xylene.

47. A process as claimed in claim 24 wherein the solvent of step (a) is toluene.

48. A process as claimed in claim 24 wherein the solvent used in step (a) is selected from the group consisting of high molecular weight ethers, polyethers comprising glycol polyethers, and cyclic ethers comprising diethyl ether, methyl-t-butyl ether, ethyl-t-butyl ether, preferably, methyl t-butyl ether.

49. A process as claimed in claim 24 wherein the solvent used in step (a) is selected from the group consisting of amides and sulpholanes.

50. A process as claimed in claim 24 wherein the solvent used in step (a) comprises a chlorinated solvent selected from the group consisting of dichloromethane, dichloroethane, chlorobenzene and dichlorobenzene.

51. A process as claimed in claim 24 wherein the solvent used in step (a) is selected from the group consisting of an alcohol, a ketone, an ester and mixtures thereof.

52. A process as claimed in claim 24 wherein the solvent used in step (a) comprises a solvent blend selected from the group consisting of tetrahydrofuran/toluene, tetrahydrofuran/heptane, and t-butylalcohol/hexane.

53. A process as claimed in claim 24 wherein the solvent used in step (a) is a mixture of a non-miscible hydrocarbon solvent comprising toluene and an aqueous solvent comprising water, enabling the separation of products obtained in aqueous phase during the course of the reaction.

54. A process as claimed in claim 24 wherein the hydroformylation reaction in step (a) is carried out at a temperature of about 140° C., preferably 90° C. to 130° C., most preferably 100° C. to 120° C., and at a pressure within the range of about 500 to about 5000 psi, preferably about 1000 to about 3500 psi.

55. A process as claimed in claim 24 wherein the hydroformylation reaction in step (a) is carried out at a temperature in the range of 90° C. to 130° C.

56. A process as claimed in claim 24 wherein the hydroformylation reaction in step (a) is carried out at a temperature in the range of 100° C. to 120° C.

57. A process as claimed in claim 24 wherein the hydroformylation reaction in step (a) is carried out at a pressure in the range of 500 to 5000 psi.

58. A process as claimed in claim 24 wherein the hydroformylation reaction in step (a) is carried out at a pressure in the range of 1000 to 3500 psi.

59. A process as claimed in claim 24 wherein step (a) is carried out at temperature in the range of 90 to 120° C.

60. A process as claimed in claim 24 wherein the hydrogen and carbon monoxide are introduced in the reaction in a molar ratio in the range of 1:2 to 8:1, preferably 1.5:1 to 5:1.

61. A process as claimed in claim 24 wherein CO and H₂ are used in step (a) in a ratio in the range of 4:1 to 1:4.

62. A process as claimed in claim 24 wherein the rhodium catalyzed hydroformylation of vinyl carboxylate includes a phosphorous, nitrogen, arsine or stilbine containing ligand selected from the group consisting of monodentate ligands such as triphenyl phosphine, tributyl phosphine, triphenyl amine, triphenyl arsine, triethyl amine, tricyclohexyl amine, pyridine, substituted pyridines such as 4-methyl pyridine and polydented ligands such as diphenyl phosphinoethane, diphenyl phosphinopropane, diphenyl phosphinobutane, diphenyl phosphinopentane, bipyridine, terpyridine.

63. A process as claimed in claim 24 wherein the reaction is carried out in the presence of an optically active ligand in order to obtain optically active 2-acetoxypropanal.

64. A process as claimed in claim 24 wherein the ratio of the ligand is at least one equivalent of ligand per mole of metal in the catalyst.

65. A process as claimed in claim 24 wherein water is added in step (b) as to provide a water: hydroformylated mixture ratio in the range of 1:1 to 1:20, preferably 1:5 to 1:15.

66. A process as claimed in claim 24 wherein the water extraction is carried out at a temperature in the range of 25° C. to 55° C. and under 50 to 300 psi carbon monoxide.

67. A process as claimed in claim 24 wherein the hydroformylation is carried out using reverse biphasic system in continuous mode, wherein water is added as a co-solvent during the hydroformylation in step (a) to create a separate phase thereby enabling extraction during the hydroformylation step.

68. A process as claimed in claim 24 wherein the aqueous extract containing acetoxypropanals obtained in step (c) is passed through a bed of purifying agents.

69. A process as claimed in claim 68 wherein the purifying agent is purifying carbon.

70. A process as claimed in claim 24 wherein the hydrogenation of the acetoxypropanals in step (c) to acetoxypropanols is carried out in aqueous solution at an elevated temperature of at least about 40° C., generally in the range of 50° C. to 175° C. and under a hydrogen pressure of at least about 100 psi generally in the range of 200 to 2000 psi.

71. A process as claimed in claim 24 wherein the hydrogenation is carried out in the presence of a hydrogenation catalyst selected from the group consisting of group VIII metals based catalysts selected from Ni, Co, Ru, Pt, Pd based catalysts and Cu, Zn, Cr, Au based catalysts including bulk, supported and fixed bed forms.

72. A process as claimed in claim 24 wherein the hydrogenation catalyst of step (c) is a supported catalyst selected from the group consisting of ruthenium, nickel, cobalt, platinum and palladium catalyst.

73. A process as claimed in claim 24 wherein the temperature of the hydrogenation reaction of step (c) is in the range of 40° C. to 90° C.

74. A process as claimed in claim 24 wherein the hydrogenation catalyst separated in step (c) is recycled.

75. A process as claimed in claim 24 wherein the hydrogenation reaction of step (c) is carried out in a continuous manner.

76. A process as claimed in claim 24 wherein the hydrogenation catalyst in step (c) is Ru/Al₂O₃.

77. A process as claimed in claim 24 wherein the hydrogenation catalyst in step (c) is Raney Ni.

78. A process as claimed in claim 24 wherein the hydrogenation is carried out in the same solvent of hydroformylation, after removal of the hydroformylation catalyst by complexation or using activated charcoal/activated silica.

79. A process as claimed in claim 24 wherein the hydrogen pressure of step (c) is in the range of 200 psig to 800 psig.

80. A process as claimed in claim 24 wherein the hydrolysis of step (d) is carried out in the presence of an acid or base catalysts for ester hydrolysis, selected from the group consisting of mineral acids, sulphonic acids, ion exchange resins, solid acids comprising ZSM-5 etc., heteropolyacids, carboxylic acids, sodium hydroxide and potassium hydroxide.

81. A process as claimed in claim 24 wherein the temperature of the hydrolysis reaction for an ion exchange resin catalyzed hydrolysis reaction is in the range of from room temperature to 120° C., preferably from 50° C. to 800° C.

82. A process as claimed in claim 24 wherein the ion exchange resin used is Amberlite IR-120cation exchange resin for the hydrolysis of a mixture of 2- and 3-acetoxypropanols to 1,2- and 1,3-propanediols.

83. A process as claimed in claim 24 wherein the hydrolysis in step (d) is carried out with 10% HCl.

84. A process as claimed in claim 24 wherein the hydrolysis step (d) is carried out in a continuous manner.

85. A process as claimed in claim 24 wherein the hydrolysis catalyst from step (d) is activated and recycled.

86. A process as claimed in claim 24 wherein the carboxylic acid, 1,3-propanediol, and 1,2-propanediol formed are separated by fractional distillation in step (e).

87. A process as claimed in claim 24 wherein the hydrolysis in step (d) is carried out before hydrogenation of step (c) in order to obtain 2- and 3- hydroxy propanal.
